(19) United States
(12) Patent Application Publication
(10) Pub. No.: US 2023/0210697 A1
(43) Pub. Date: Jul. 6, 2023

(54) Voltaic Arc Processing Apparatus and Method

(71) Applicant: EPS.SYSTEMS SRL, Genova (IT)

(72) Inventors: Mario D'Aquino, Albissola Marina (IT); Gianluca D'Aquino, Godiasco Salice Terme (IT)

(21) Appl. No.: 18/000,767

(22) PCT Filed: Jun. 4, 2021
(86) PCT No.: PCT/IB2021/054898

§ 371 Date: Dec. 5, 2022

(30) Foreign Application Priority Data
Jun. 5, 2020 (IT) 102020000013351

(51) Int. Cl. A61F 9/06; H04N 7/18; H04N 23/69; H04N 23/23

(52) U.S. Cl. CPC A61F 9/065; H04N 7/18; H04N 23/69; H04N 23/23; H04N 23/75

(57) ABSTRACT

A voltaic arc processing apparatus and method are described for processing metal workpieces, like MMA electrode welding, TIG/MIG/MAG welding, plasma welding, plasma cuts, etc., in which a welding mask includes a protective head covering worn by an operator to protect eyesight from a very intense light emission produced during processing, two or more cameras arranged outside the protective head covering to send the images of the processing to a screen arranged inside the protective head covering and visible by the operator, wherein the operator can set one or more operating parameters of the processing that are displayed on the screen.

### TECHNICAL FIELD

The invention relates to a processing apparatus and method in which a very intense luminous emission is produced. In particular, reference is made to an apparatus and a method that enables the sight to be protected of an operator assigned to processing, for example voltaic arc processing.

### BACKGROUND

Specifically, but not exclusively, the invention in question can be usefully used in a voltaic arc apparatus and/or method for performing processing on metal workpieces, such as, for example, electrode welding (MMA), TIG/MIG/MAG welding, plasma welding, plasma cuts, etc.

Patent publication US 2019/0083316 A1 shows a welding mask with a lamp to provide the operator with illumination.

Patent publication US 2007/0079417 A1 shows a mask for welding with an LCD screen.

Various aspects are improvable of known processing system in which a very intense luminous emission is produced, for example voltaic arc systems, in particular for welding with MMA, TIG, MIG, IMAG etc processes.

It is firstly desirable to increasingly ensure the health and safety of the operator.

The prior art involves protecting from ultraviolet radiation, in general by the use of covering for the body and hand and of a mask with a self-darkening filter for the eyes. It is nevertheless known that a self-darkening filter moves from the transparent state to the darkened state with a certain latency time, which is very short but is not nil, for example a lapse of time comprised between about 0.4 mS, for more performing filters, and beyond 1 mS, for more poorly performing filters.

This means that the operator, each time he turns on the arc, receives a peak of UV rays in the eyes for a certain time (as said, from a minimum of 0.4 mS to more than 1 mS). In some types of processing, in particular in the case of welding by points (making small but numerous welds on large surfaces, for example to keep the metal sheets stationary before final fixing), the operator has to perform very brief welding tasks, for example from 1 to 5 seconds, but repeated over time. In these cases, the eyes of the operators can receive a very high number of UV ray peaks, because of the aforesaid latency time of the self-darkening filter.

It is also desirable to overcome another drawback that is due to the fact that the self-darkening filter of the welding mask, when it is open, i.e. transparent, is noticeably darker than the view without mask. For this reason, the operator, when working in an environment with poor lighting, is forced to lift the mask, adjust the workpieces, lower the mask again, start welding and possibly repeat operations if positioning is not perfect.

Another drawback is linked to the fact that prior art welding apparatuses usually use a user interface having a screen that should permit the processing parameters to be displayed and/or set (for example, welding current, speed of supply of the welding material, current ascent and descent ramps, etc). Nevertheless, through the effect of the polarized light, the screens used are not very visible through the visor of the mask (also with the filter in a transparent condition), so the operator, risking damage to his sight, may set the parameters with the visor raised and, still with the visor raised, starts a short welding portion (with the sole precaution of not looking directly at the arc), makes further adjustments, and, lastly, when the setting of the parameters is complete, lowers the visor and starts welding proper. This operating mode is not permitted by regulations and exposes the operator to a high quantity of UV rays.

### SUMMARY

One object of the invention is to provide a solution to one or more of the aforesaid limits and drawbacks of the prior art.

One object is to provide a processing apparatus and/or method that are alternative to the prior art, to protect an operator from a very intense light emission produced during processing.

One advantage is making a mask that is wearable by an operator to protect the operator effectively from very intense light emissions, in particular from ultraviolet radiation generated during processing.

One advantage is to reduce the risk that the operator does not wear the protective mask correctly donned during emission of the radiation.

One advantage is to permit an operator to see the processing zone appropriately and easily, also operating in an area with poor lighting and with the protective mask worn correctly.

One advantage is to enable a user interface to be used conveniently and appropriately with a screen, in particular for displaying and/or setting the processing parameters (for example displayed superimposed on the images of the processing zone that are taken by a camera , as will be explained better below), also with the mask worn correctly in a safe condition.

One advantage is to make available a constructionally simple and cheap voltaic arc processing apparatus.

These objects and advantages and still others are achieved by an apparatus and/or a method according to one or more of the claims set out below.

In one embodiment, a processing apparatus includes a mask to protect eyesight from a very intense luminous emission, a camera arranged on the mask to detect images of the processing zone, a screen arranged inside the mask to enable the operator wearing the mask to see on the screen the images received from the camera.

The processing apparatus may include a control device for displaying on the screen one or more processing operating parameters. The processing apparatus may include a control device, connected operationally to the screen element, to enable the operator to set one or more processing operating parameters.

In one embodiment, one processing method includes the steps of detecting the images of a processing task performed by an operator wearing a protective mask, the images being detected by a camera arranged on the mask, and of displaying the images detected by the camera on a screen arranged inside the mask, so that the operator wearing the mask can see the images on the screen.

The method may include the step of displaying on the screen information on one or more processing operating parameters.

### BRIEF DESCRIPTION OF THE DRAWINGS

The invention can be better understood and implemented with reference to the attached drawings that illustrate some embodiments thereof by way of non-limiting example, in which:

FIG. 1 shows a frontal view of one embodiment of a protective mask made according to the invention;

FIG. 2 is a side view of the mask of FIG. 1;

FIG. 3 is a view of the inside of the mask of FIG. 1;

FIG. 4 is a schematic view of one embodiment of a processing apparatus made according to the invention;

FIG. 5 is a schematic side view of the embodiment of FIG. 4.

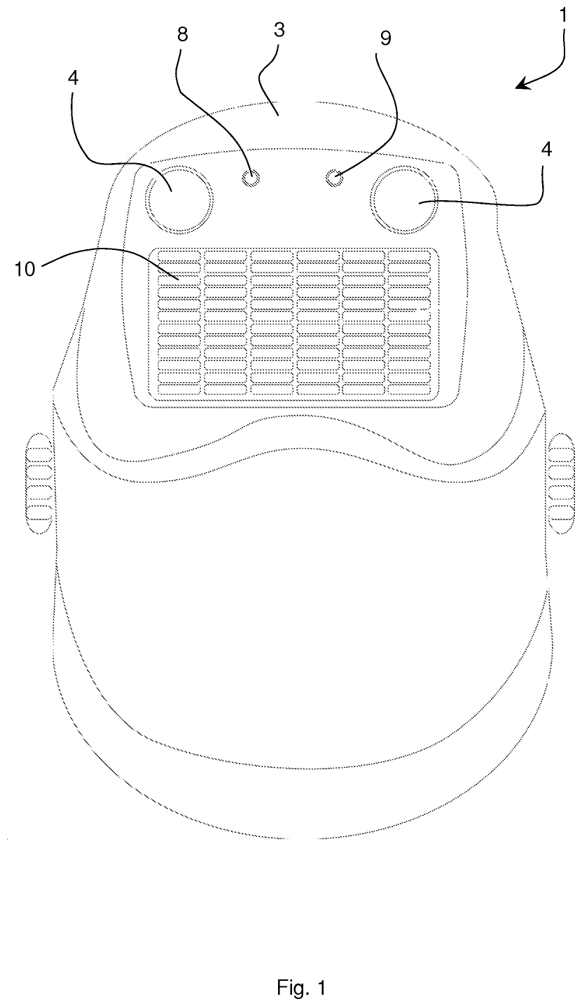

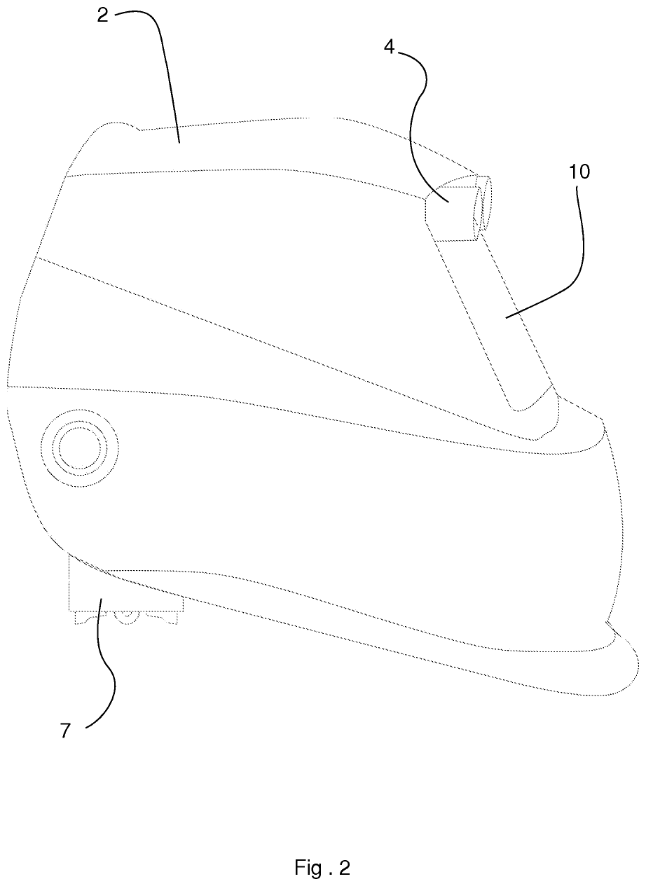

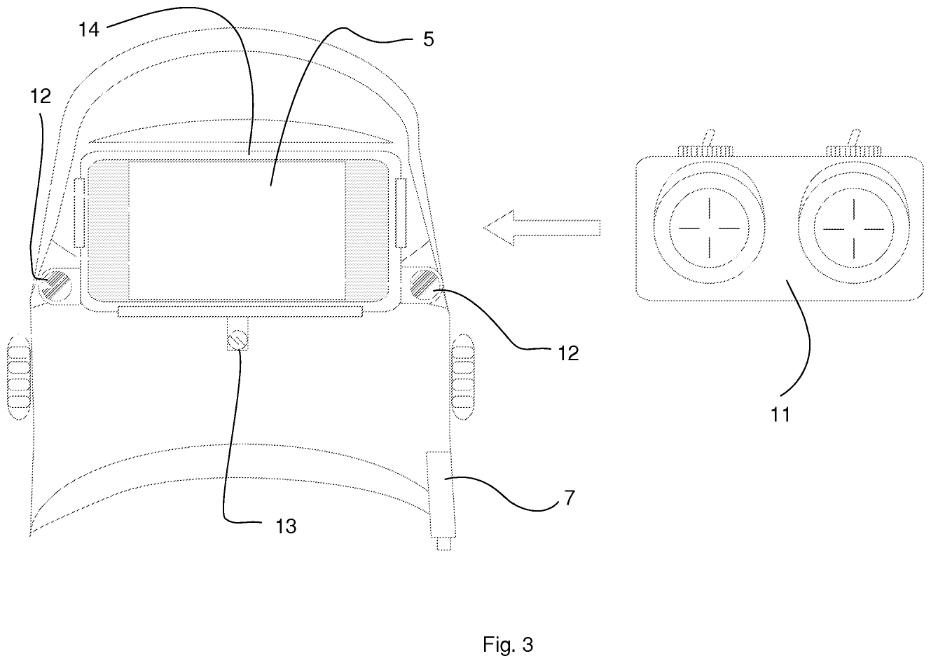

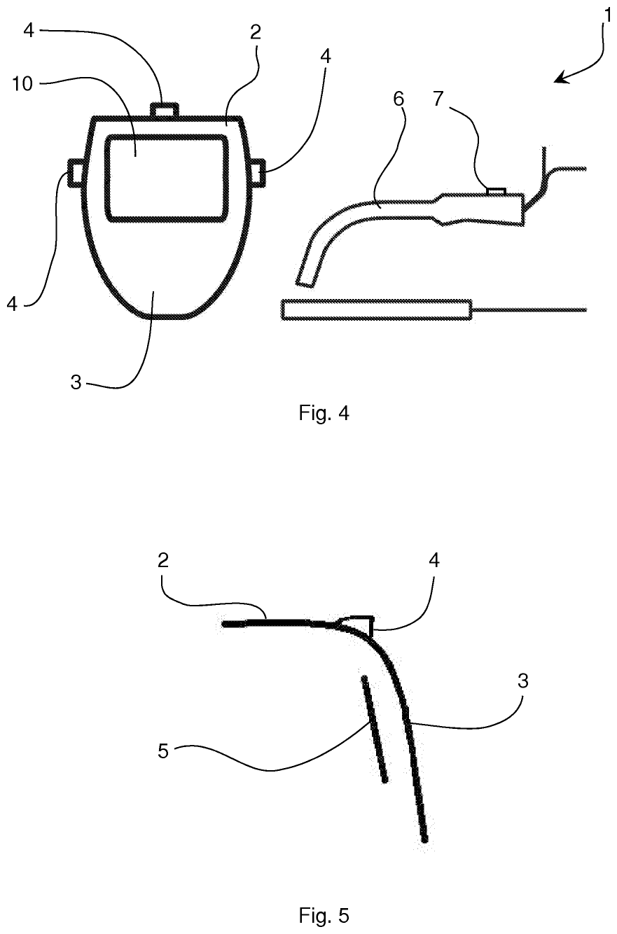

### DETAILED DESCRIPTION

For clarity of exposition, analogous elements of different embodiments have been indicated by the same numbering.

With reference to the aforesaid figures, 1 indicates overall a processing apparatus, in particular for performing processing in which a very intense luminous emission is produced.

The apparatus 1 may include, in particular, an apparatus for voltaic arc processing. The apparatus 1 may be configured, in particular, to perform tasks on metal workpieces, such as, for example, electrode welding (MMA), TIG/MIG/MAG welding, plasma welding, plasma cuts, etc.

The apparatus 1 may include, in particular, a welding mask 2 including a protective head covering 3 to protect the eyesight of an operator from a very intense light emission produced during processing, for example a protective head covering 3 suitable for protecting eyesight from ultraviolet radiation.

The apparatus 1 may include, in particular, at least one camera 4 arranged outside the protective head covering 3.

The apparatus 1 may include, in particular, a protective head covering 3 that is completely closed, totally opaque and impermeable to the passage of light radiation. The camera 4 may be arranged, in particular, on the protective head covering.

The apparatus 1 may include, in particular, one or more further cameras 4 arranged outside the protective head covering 3. The further cameras 4 may be arranged on the protective head covering 3. The apparatus 1 may include, in total, two, or three, or four cameras 4, even if it is possible to provide a different number of cameras.

The apparatus 1 may include, in particular, at least one screen 5 arranged inside the protective head covering 3. The screen 5 may be conformed and arranged so as to be visible to a person wearing a protective head covering 3. The screen 5 may be configured to display the images detected by the camera/s 4. The apparatus 1 may include, in particular, one or more further screens arranged inside the head covering 3.

The apparatus 1 may include, in particular, a control device configured to enable an operator to set one or more processing operating parameters. The apparatus 1 may include, in particular, a control device configured to display on the screen 5 information on one or more processing operating parameters. The aforesaid settable and/or displayable processing operating parameters may include, in particular, the welding current, the welding material supply speed, the current ascent and descent ramps, etc.

The apparatus 1 may include, in particular, a device for adjusting at least one feature of the screen 5 in function of at least one feature of the images detected by the camera/s 4. The device for adjusting may be configured, in particular, to adjust a photometric parameter of the screen 5 as a function of a photometric parameter of the images. The aforesaid photometric parameter of the screen 5 may include, in particular, light intensity. The aforesaid photometric parameter of the images may include, in particular, light intensity.

The apparatus 1 may include, in particular, at least one voltaic arc generator 6 connected to the control device and configured to performing the processing task. The voltaic arc generator 6 may include, in particular, a voltaic arc torch, for example a torch configured to weld.

The apparatus 1 may include, in particular, a zoom device configured to enlarge the image displayed on the screen 5 and/or widen the field of view of the camera 4 and/or to extend the field of view of each of the further cameras 4.

The apparatus 1 may include, in particular, at least one heat camera arranged outside the protective head covering, for example arranged on the protective head covering. The heat camera may be connected to the control device, which may be configured to display on the screen the information supplied by the heat camera during processing, for example information on the temperatures in the processing zone.

The control device may include, in particular, at least one computer and at least one input device connected to the computer. The input device 7 may include, in particular, at least one end control portion that is operatable by hand by the operator wearing the mask 2. The end control portion may be arranged, in particular, on the voltaic arc torch, so that an operator can operate the end control portion whilst grasping the voltaic arc torch that he uses to perform the task. The end control portion may be arranged, in particular, on the head covering 3, or may be a movable element, for example a wearable electronic device, or a device configured with a remote control function.

The apparatus 1 may include, in particular, a light intensity sensor 8 arranged (in particular outside the protective head covering 3) to detect the light intensity of the processing zone in which the intense light emission is generated.

The apparatus 1 may include, in particular, at least one microphone 9 arranged on the mask 2.

The apparatus 1 may include, in particular, an optoelectronic device 10 for supplying the apparatus 1, in particular supplying the various electrically powered devices of the apparatus, such as for example the screen 5 and the cameras 4. The optoelectronic device 10 may include, in particular, at least one photovoltaic module. The optoelectronic device 10 may be configured, in particular, to convert light energy into electric energy.

The apparatus 1 may include, in particular, one or more focus lenses 11 of the cameras 4.

The apparatus 1 may include, in particular, one or more loudspeakers 12 arranged on the head covering 3 or in the head covering 3.

The apparatus 1 may include, in particular, at least one inner microphone 13 arranged inside the head covering 3.

The apparatus 1 may include, in particular, a pointer (which is not shown, for example, a laser pointer) arranged for enabling the operator (welder) to point at a point of the processing zone (for example a desired point of the workpiece/s to be welded). The pointer may be arranged, in particular, on the mask 2. The pointer may be used, in particular, to obtain a temperature measurement (from a heat camera) in the desired point of the processing zone.

The apparatus 1 may include, in particular, at least one optical filter arranged on each camera 4 and configured to attenuate the light intensity of the images detected by the camera/s 4. The apparatus 1 may include, in particular, at least one software filter configured to regulate the contrast of the images detected by the camera/s 4 and displayed on the screen 5.

The apparatus 1 may include, in particular, a GPS positioning device. The control device may be configured, in particular, to store in a memory the images detected by the camera/s 4, together with the information supplied by the GPS positioning device, possibly used with the process parameters set and used for each process.

The control device may be configured, in particular, to associate the images stored with the corresponding positioning information and/or with the corresponding stored process parameters, so as to construct a database that is usable to identify and analyze some of the main features of the various processes performed with the apparatus 1 at different times and in different places.

The operation of the processing apparatus 1 can implement a processing method that may include, in particular, the step, performed to prepare for the processing task proper, of providing a mask 2 with a protective head covering 3 configured to protect an operator wearing the protective head covering 3 from a very intense light emission produced during the processing task.

The processing method may include, in particular, the step, performed during processing proper, of detecting, by one or more cameras 4 arranged outside the protective head covering 3, the images of the processing performed by the operator wearing the mask 2.

The processing method may include, in particular, the step of displaying the images detected by the cameras 4 on a screen 5 arranged inside the head covering 3, so that the operator wearing the protective head covering 3 can see the images on the screen 5.

The processing method may include, in particular, the step of displaying on the screen 5 the information relating to one or more processing operating parameters, in particular operating parameters set by the operator.

As has been seen, the processing apparatus 1 may include, in particular, a welding system provided with a protective mask 2 for the welder and further provided with a data collection system that enables a facilitated process to be provided in real time for validating welding processes, in which the collected welding data include at least the welding parameters, the date of performance of the welding tasks, the image of the welding tasks performed, etc.

The welding mask 2 may include, in particular, a protective head covering 3 that may be completely closed to safeguard the operator completely, configured so that the operator will never be induced to remove or open the protective head covering 3 until the light emission of the voltaic arc has ceased definitively.

The welding process will be able to be observed, in particular, by a plurality of cameras 4, each with an observation point, significantly improving the possibility to view and inspect the process.

The mask 2 may include, in particular, at least one hardware filter for lowering the light intensity of the image (in particular a solar filter, like for example a metalized glass plate or a transparent polymeric sheet) and/or at least one software filter to increase the image contrast.

It is possible to monitor and/or observe the welding process by a heat camera, in particular to obtain information (possibly displayed on the screen) on the heat distribution in the welded workpiece.

Owing to the invention, the operator (welder) can observe the welding process on the screen 5 (for example a colour LCD monitor) positioned at a short distance from the eyes, without ever looking towards the welding zone and without ever being induced to raise or remove the head covering 3. In this manner, the eyes of the operator will never be exposed directly to the ultraviolet rays.

The control device is so configured as to adjust the image shown on the screen 5 in function of the effective light intensity in the welding zone, in particular attenuating automatically the light intensity of the image shown on the screen 5 when the electric arc is switched on.

The control device may be configured, in particular, so as to increase the light intensity of the image shown on the screen 5 in function of the actual light intensity in the place and in the welding zone, for example when the electric arc is switched off and the environment where the welder works is poorly lit.

For this purpose, the light intensity sensor 8 may be used to supply to the control device the signals indicating the actual light intensity in the place and in the welding zone.

The control device may be so configured, in particular, that before and/or during and/or after machining, on the screen 5 one or more setting parameters of the apparatus 1 are displayed (for example superimposed in a corner of the screen 5), so that the operator can observe the welding zone on the screen 5 and at the same time, substantially without diverting his gaze from the workpiece being processed, can read the operating data that appear on the screen 5. It is possible for menus to be displayed on the screen that may be activated and browsed, in particular by the input device 7.

The end control portion may include, in particular, an encoder or a wearable electronic device connected to the apparatus (for example by a wireless connection, by Bluetooth, radio waves, or another device). The end control portion may include, in other embodiments, a remote control, a joystick, a switchboard, a mouse, a touchpad, etc. The end control portion may be arranged, for example, on the body of the voltaic arc torch, or on the protective head covering, or may be worn by the user, or may be arranged elsewhere.

The processing apparatus 1 may be equipped with a zoom control to enable the observed object to be enlarged (the workpieces in the welding zone) or the field of view of the operator to be expanded.

In the case of welding used to make particularly costly products or subjected to critical stress, it is often necessary to certify every single weld, such as, for example, in the case of construction of a boat, or of conduits containing hazardous liquids, etc. In these cases, once the processing task has been performed, every single weld has to be inspected, in particular by visual examination, radiography, etc, to ensure the conformity of the welding process performed and the absence of defects that may compromise the mechanical resistance thereof.

The system disclosed here can facilitate the process of certification of the welding performed by providing significant data. The (electronic and programmable) control device of the mask and of the voltaic arc generator may be configured to save the videos of what the operator sees.

The control device may include, in particular, a connecting device for connecting to a telecommunications network (for example the internet) and/or a GPS device. Each video of a welding task may be saved with the geographical coordinates of the place where the welding took place. The stored data on each video of a welding task may include one or more parameters of the corresponding welding process, like for example the current used in the process and/or the type of wire used (in the case of a MIG process). The welding wire may be supplied on coils that include an identification (for example an RFID device) that enables the material of the wire, the weight of the complete coil and other parameters to be recognized and certified. The stored data on each video of a welding task may include, in particular, one or more parameters regarding the operator, like, for example, heartbeat and/or blood alcohol level and/or the inner temperature of the mask, etc.

It is possible to provide certification of the date on which the film was taken by the camera/s of the apparatus, in particular by access, via the network, to a validation service of the welding process (for example by blockchain, or another type of data structure). The apparatus in question enables a video archive to be generated of the images that the welder saw during his work, with the possibility of associating each welding task with the place and date of performance of the welding task, and possibly with some welding parameters (welding current and/or speed of the welding wire and/or type of wire and/or monitoring of the flow of the protection gas and/or monitoring of the electric supply and/or still other parameters). The specific data on each welding task are associable with the films of the relative welding task, which enables certification of the welding process to be greatly simplified.

It is further possible to evaluate the profitability of the work done by the operator, for example by collecting the videos of the work done by collecting the videos of the work done (at the end of the day or in real time), which videos are stored and associated with the masks worn by the various operators, to then analyze each video together with the relative operating parameters of the work done, such as for example the consumption of the welding material, power consumption, the length of time taken for the task and yet other parameters.

The control device of the processing apparatus 1 may include, in particular, a management device configured to manage a plurality of user profiles. This management device may be activatable, in particular, with biometric data, for example by a facial recognition device or a fingerprint reader, to certify the identity of the operator. The mask 2 may include, in particular, an inner chamber 14 for facial recognition of the operator.

The mask 2 may be provided, in particular, with a signalling device (for example fitted to the mask 2). This signalling device may include, in particular, at least one luminous and/or acoustic signalling device. In particular, the luminous signalling device may be activatable in different colours and may be configured to indicate, with different colours the different states of activity during the course of processing.

The control device may include a diagnostic device configured to switch off immediately the apparatus in the event of a fault.

The processing apparatus 1 may include, in particular, a movement and/or position sensor, for example arranged on the welding torch, operationally connected to the voltaic arc generator.

1. A voltaic arc processing apparatus, comprising:
a mask to protect an operator from a very intense light emission produced during a voltaic arc process;
at least one camera on said mask;
a screen inside said mask to be visible by a wearer of said mask and configured to display images detected by said at least one camera.

2. A voltaic arc processing apparatus according to claim 1, including a control device configured to allow an operator to set one or more operating parameters of said process and/or a control device configured to display information relating to one or more operating parameters of said process on said screen.

3. A voltaic arc processing apparatus according to claim 1, including a device for adjusting at least one feature of said screen as a function of one or more features of images detected by said at least one camera.

4. A voltaic arc processing apparatus according to claim 3, wherein said device for adjusting is configured to adjust a photometric quantity, in particular a light intensity, of said screen as a function of a photometric quantity, in particular a light intensity, of said images; said apparatus including, in particular, a light intensity sensor arranged on said mask to detect light intensity in a processing area.

5. A voltaic arc processing apparatus according to claim 2, including a voltaic arc generator connected to said control device and configured to perform said process.

6. A voltaic arc processing apparatus according to claim 2, including:
one or more further cameras on said mask; said apparatus including, in particular, a zoom device configured to enlarge said image displayed on said screen and/or to enlarge the visual field of said at least one camera and/or to enlarge the visual field of each of said one or more further cameras; and/or
at least one thermal camera on said mask and connected to said control device which is configured to display on said screen information provided by said thermal camera.

7. A voltaic arc processing apparatus according to claim 4, including a pointer on said mask and configured to point a desired point of the processing area and obtain a temperature measurement from a thermal camera in the desired point of the processing area; said pointer including, in particular, at least one laser pointer.

8. A voltaic arc processing apparatus according to claim 2, wherein said control device includes at least one electronic processor and at least one input device connected to said processor; said input device including, in particular, at least one terminal control portion which can be operated by hand by the operator wearing the mask; said apparatus including, in particular, a voltaic arc terminal element, for example a torch, operatively connected with said terminal control portion; said terminal control portion being arranged on said terminal element, or being a movable portion, for example arranged on a wearable device.

9. The voltaic arc processing apparatus according claim 1, including:
a recognition device arranged on said mask and configured to identify the wearer of said mask; and/or
a device for connecting said apparatus to a telecommunications network and including, in particular, a Bluetooth connection card and/or a network card and/or a GPS or satellite communication device.

10. The voltaic arc processing apparatus according to claim 1, including an optoelectronic device, in particular of the photovoltaic type, arranged on said mask to supply at least one device of said apparatus.

11. The voltaic arc processing apparatus according to claim 2, including:
at least one optical filter on said at least one camera configured to attenuate light intensity of the images detected by said at least one camera and/or at least one software filter configured to adjust contrast of the images displayed on said screen; and/or
a GPS positioning device, said control device being connected to said GPS positioning device and being configured to store in a memory images detected by said at least one camera, information provided by said GPS positioning device, and set process parameters, so that said stored images are associated with the corresponding positioning information and the corresponding set process parameters.

12. A processing method, including the steps of:
before processing, providing a mask configured to protect an operator wearing the mask from a very intense light emission produced during processing;
during processing, detecting images of the processing performed by the operator wearing the mask by at least one camera on said mask;
displaying said images detected by said at least one camera on a screen-inside said mask so that the operator wearing the mask can see said images on said screen;
displaying on said screen information relating to one or more operating parameters of said processing.
